(19) United States
(12) Patent Application Publication
(10) Pub. No.: US 2009/0205110 A1
(43) Pub. Date: Aug. 20, 2009

(54) Earmuff device

(75) Inventor: Tsung-Pai Chiang, Taipei City (TW)

Correspondence Address: BACON & THOMAS, PLLC, 625 SLATERS LANE, FOURTH FLOOR, ALEXANDRIA, VA, 22314-1176, US

(73) Assignee: SOUND TEAM ENTERPRISE CO., LTD., Taipei City (TW)

(21) Appl. No.: 12/216,278

(22) Filed: Jul. 2, 2008

(30) Foreign Application Priority Data
Feb. 15, 2008 (TW) 097105463

(51) Int. Cl. A61F 11/14

(52) U.S. Cl. 2/209

(57) ABSTRACT

An earmuff device is disclosed to include a headpiece formed of two smoothly arched bands that are overlapped on each other and axially slidable relative to each other, two earmuff cups each having a cup base, a gradual enlargement cup rim and a plurality of radial ribs radially connected between the cup base and the gradual enlargement cup rim, two U-lugs respectively pivotally connected to the bands of the headpiece, two connecting strips each having one end pivoted to one U-lug and the other end fastened to one earmuff cup, two earmuff cushions respectively covering the earmuff cups, and a soft sleeve sleeved onto the headpiece and connected between the earmuff cushions. The earmuff device allows a user to conveniently adjust the length of the headpiece as well as the angular position of the headpiece relative to the earmuff cups.

This application claims the priority benefit of Taiwan patent application number 097105463 filed on Feb. 15, 2008.

### BACKGROUND OF THE INVENTION

1. Field of the Invention

The present invention relates to earmuffs and more particularly, to an earmuff device that allows the wearer to conveniently adjust the length of the headpiece as well as the angular position of the headpiece relative to the earmuff cups. Further, the earmuff device can be received in a collapsed manner with the two earmuff cups attached together to save storage space.

2. Description of the Related Art

In winter or cold places, people usually wear heavy clothes, muffler and gloves to keep the body and the hands warm. When walking in a high mountain, snow land and outdoors during a winter season, regular clothes cannot well protect the ears, and the ears may ache or may be affected by frostbite. For keeping the ears warm, earmuffs shall be used.

FIG. 16 illustrates a conventional earmuff device. According to this design, the earmuff device comprises an adjustable headpiece A that is formed of two bands axially slidably coupled to each other, and two earmuffs B respectively fastened to the two distal ends of the headpiece A with a respective fastening member C. When in use, the headpiece A is adjusted to the desired length, and then the two earmuffs B are respectively attached to the ears. This design of earmuff device is still not satisfactory in function because of the following drawbacks:

1. The earmuff device can be worn on the user's head from the top or back side in one single direction.

2. The protruding ring end of each band of the headpiece A is kept in contact with the wearer's head, causing the wearer to feel uncomfortable.

3. Because the two bands of the headpiece A have a uniform transverse thickness, the headpiece A may be twisted easily. Further, because the angular position of the earmuffs B is not adjustable relative to the headpiece A, the earmuff device may fall from the wearer's head easily by an external force.

4. Because the earmuff device simply utilizes the small fastening member C to secure the earmuffs B to the headpiece A, the connection area between the earmuffs B and the headpiece A may break easily when the wearer adjust the position of the earmuffs B.

5. The earmuffs B have low structural strength and low grasping force. In consequence, the earmuffs B cannot cover the wearer's ears positively.

6. When the earmuff device is not in use, the user cannot receive the earmuff device in a collapsed condition with the earmuffs B attached together to save storage space. When receiving the earmuff device, the earmuff device may be broken accidentally.

7. The earmuff device is packed in the assembled condition. Therefore, this design does not allow the headpiece and the earmuffs to be separately wrapped with a respective fabric covering.

### SUMMARY OF THE INVENTION

The present invention has been accomplished to provide an earmuff device that eliminates the aforesaid drawbacks. According to one aspect of the present invention, the earmuff device is comprised of a headpiece, two earmuff cups, two connecting devices respectively connecting the earmuff cups to the headpiece, and two earmuff cushions respectively covered on the earmuff cups. The headpiece is comprised of two smoothly arched bands that are overlapped on each other and axially slidable relative to each other. Each band has a coupling block disposed at one end and defining an insertion slot, and two sliding grooves bilaterally extending along the length. One band is inserted through the insertion slot of the other band and the coupling block of one band is slidably coupled to the sliding grooves of the other band so that the two bands are kept in a parallel manner and can be moved relative to each other to adjust the length of the headpiece. Because the sliding grooves bilaterally extend along the length of the respective band, the two opposite lateral sides of each band are relatively thinner. Therefore, the stress is concentrated on the middle part of each band during a use of the earmuff device, preventing distortion of the earmuff device.

According to another aspect of the present invention, each connecting device comprises an U-lug pivotally coupled to one end of one band of the headpiece, and a connecting strip pivoted to the U-lug and fastened to one earmuff cup. Therefore, the user can adjust the earmuff cups forwards and backwards as well as leftwards and rightwards. Further, each band has a chamfered end edge kept in friction engagement with the associating U-lug so that the U-lug can be positively positioned in one angular position relative to the associating band.

According to another aspect of the present invention, each earmuff cup has a cup base, an insertion hole in the cup base, and at least one engagement hole cut through the cup base across the insertion hole. Further, the connecting strip of each connecting device is inserted into the insertion hole of one earmuff cup, having at least one retaining rib respectively forced into engagement with the at least one engagement hole of the associating earmuff cup.

According to still another aspect of the present invention, the earmuff cushions are respectively made in the form of a pocket member that receives the associating earmuff cup. Further, each earmuff cushion has an opening through which the connecting strip of the associating connecting device is inserted into the insertion hole of the associating earmuff cup. Further, a flexible fabric sleeve may be sleeved onto the headpiece and connected between the earmuff cushions.

According to still another aspect of the present invention, the U-lugs of the connecting devices can be turned relative to the headpiece and the earmuff cups can be turned with the associating connecting strips relative to the associating U-lugs so that the earmuff device can be received in a compact condition, saving the storage space.

### BRIEF DESCRIPTION OF THE DRAWINGS

FIG. 1 is a perspective view of an earmuff device according to the present invention (the earmuff cushions excluded).

FIG. 2 is an exploded view of FIG. 1.

FIG. 3 is a schematic drawing showing adjustment of the length of the headpiece according to the present invention.

FIG. 4 is a sectional view of a part of the present invention, showing connection between one band of the headpiece and the associating connecting device.

FIG. 5 is a sectional view of a part of the present invention, showing connection of one connecting device between the associating band and the associating earmuff cup.

FIG. 6 is a schematic drawing showing the earmuff cup turned with the associating connecting device relative to the headpiece according to the present invention.

FIG. 7 is a schematic sectional view of a part of the present invention, showing the earmuff cup turned with the connecting strip of the associating connecting device relative to the associating U-lug and the headpiece.

FIG. 8 illustrates one received status of the earmuff device according to the present invention (the earmuff cushions excluded).

FIG. 9 illustrates another received status of the earmuff device according to the present invention (the earmuff cushions excluded).

FIG. 10 is an exploded view of a part of the present invention, showing one form of the earmuff cushion.

FIG. 11 is a sectional view of a part of the present invention, showing another form of the earmuff cushion.

FIG. 12 is a perspective view of the present invention, showing the two earmuff cushions respectively covered on the earmuff cups.

FIG. 13 is a perspective view of an alternate form of the present invention, showing a fabric sleeve sleeved onto the headpiece and connected between the earmuff cushions.

FIG. 14 is a perspective view of another alternate form of the present invention, wherein the fabric sleeve is formed integral with the earmuff cushions.

FIG. 15 is a schematic drawing showing wearing adjustment of the earmuff device according to the present invention.

FIG. 16 is an elevational view of an earmuff device according to the prior art.

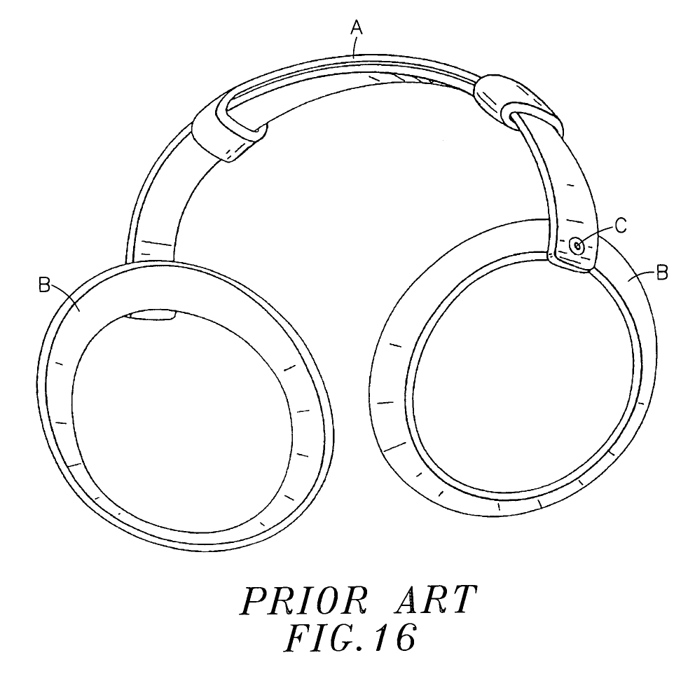

### DETAILED DESCRIPTION OF THE PREFERRED EMBODIMENT

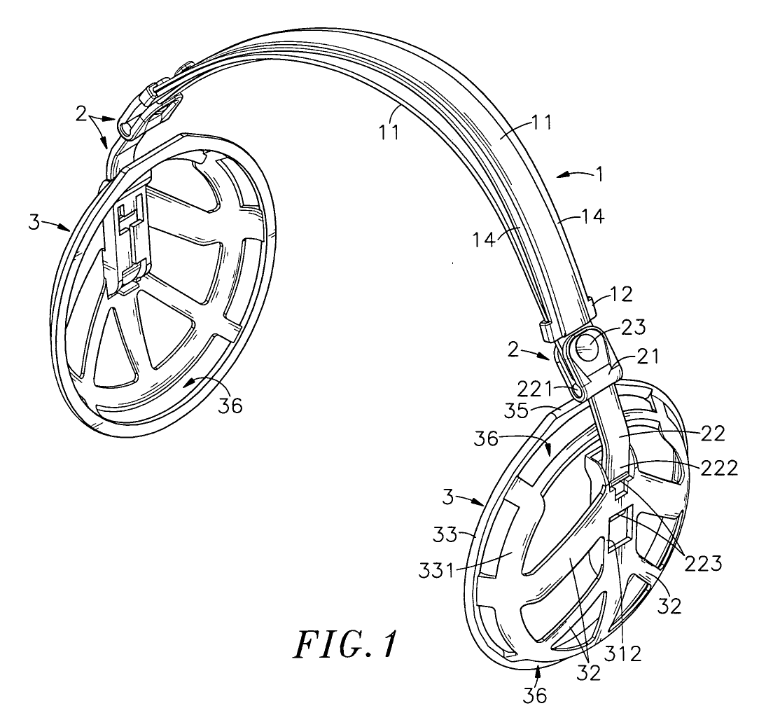

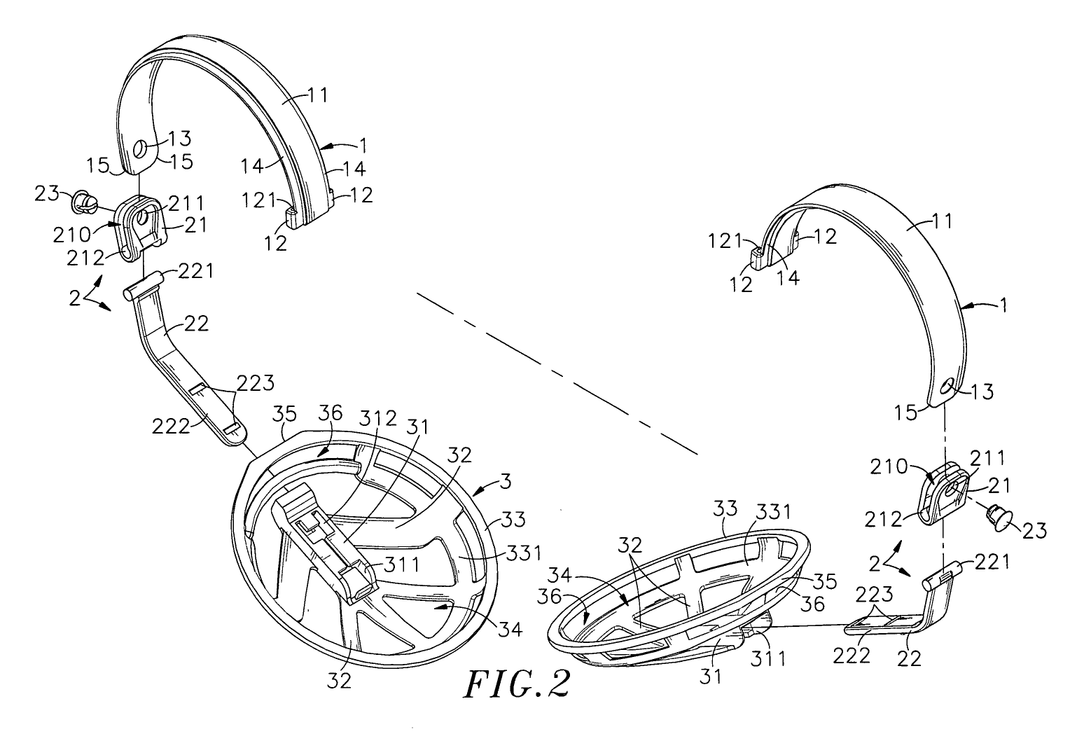

Referring to FIGS. 1, 2, 10 and 11, an earmuff device is shown comprising a headpiece 1, two connecting devices 2, two earmuff cups 3 and two earmuff cushions 4.

The headpiece 1 is comprised of two smoothly arched bands 11 that are overlapped on each other and axially slidable relative to each other. Each band 11 has a first end provided with a chamfered end edge 15 and provided with a through hole 13, a second end opposing to the first end and provided with a coupling block 12 that defines an insertion slot 121, and two sliding grooves 14 bilaterally extending along the length between the first end and the second end. By means of inserting the first end of one band 11 through the insertion slot 121 of the coupling block 12 of the other band 11, the two bands 11 are coupled together and axially slidable relative to each other to adjust the combined length, and the sliding grooves 14 of one band 11 guide sliding movement of the coupling block 12 of the other band 11.

The connecting devices 2 are adapted for securing the two earmuff cups 3 to the first ends of the two bands 11 of the headpiece 1 respectively, each comprised of a U-lug 21, a connecting strip 22 and a pivot bolt 23. The U-lug 21 has a flat receiving open chamber 210 defined between the two opposite sidewalls thereof, two through holes 211 respectively cut through the two opposite sidewalls and aligned in line at two opposite sides of the flat receiving open chamber 210, and a bottom pivot housing 212 connected between the two opposite sidewalls. The connecting strip 22 has a pivot axle 221 transversely disposed at its one end and pivotally coupled to the bottom pivot housing 212 of the U-lug 21, a connecting endpiece 222 disposed at its other end, and at least one, for example, two retaining ribs 223 transversely disposed at the connecting endpiece 222 and spaced in direction along the length of the connecting endpiece 222.

The earmuff cups 3 are respectively fastened to the connecting strips 22 of the connecting devices 2, each having a cup base 31, an insertion hole 311 defined in the cup base 31 for the insertion of the connecting strip 22 of the associating connecting device 2, at least one, for example, two engagement holes 312 cut through the cup base 31 and extending across the insertion hole 311, a gradual enlargement cup rim 33, a plurality of radial ribs 32 radially connected between the cup base 31 and gradual enlargement the cup rim 33, a plurality of smoothly arched reinforcing ribs 331 connected with the radial ribs 32, an accommodation space 34 surrounded by the radial ribs 32, the cup base 31, the cup rim 33 and the smoothly arched reinforcing ribs 331, a plurality of locating holes 36 defined between the cup rim 33 and the smoothly arched reinforcing ribs 331 and separated by the radial ribs 32.

When the headpiece 1 and the earmuff cups 3 are fastened together by the connecting devices 2, the two earmuff cushions 4 are respectively covered on the two earmuff cups 3 for contact with the side of the wearer's head.

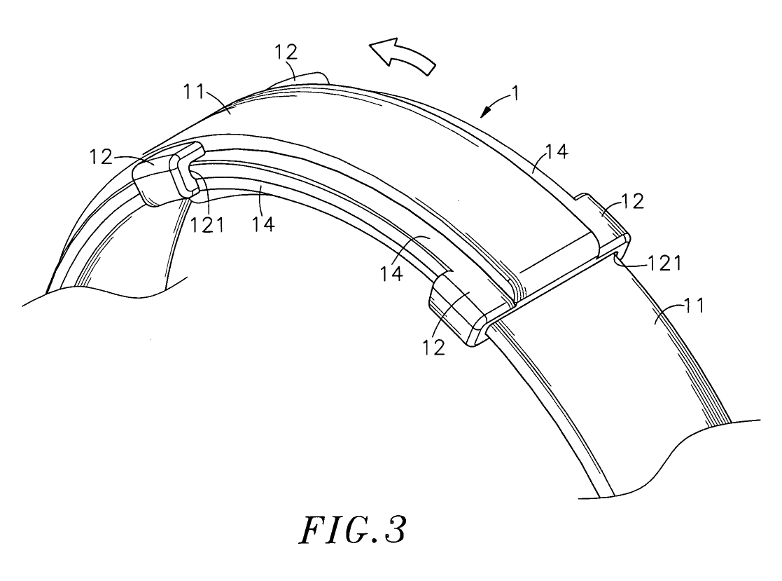

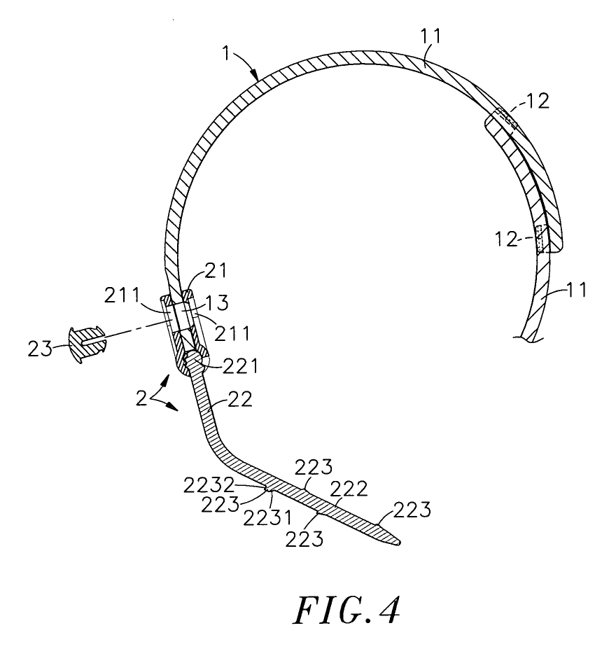

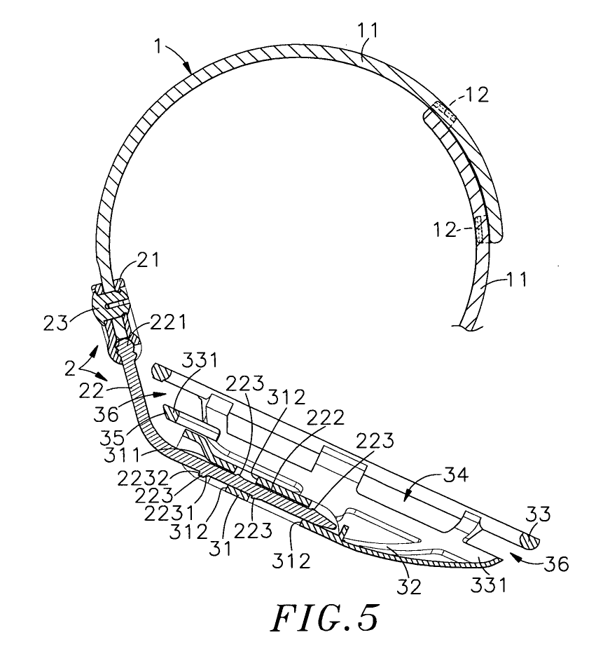

Referring to FIGS. 3˜5, the first end of one band 11 is inserted through the insertion slot 121 of the coupling block 12 of the other band 11 so that the two bands 11 are coupled together and can be moved axially relative to each other to adjust the combined length (see FIG. 3). When adjusting the length of the headpiece 1, the sliding grooves 14 of one band 11 guide sliding movement of the coupling block 12 of the other band 11. Thereafter, the pivot axles 221 of the connecting strips 22 of the connecting devices 2 are respectively pivotally coupled to the bottom pivot housings 212 of the associating U-lugs 21, and then the bands 11 are respectively inserted into the flat receiving open chambers 210 of the U-lugs 21 to have the respective through holes 13 of the bands 11 be in alignment with the respective through holes 211 of the U-lugs 21, and then the pivot bolts 23 are respectively fastened to the through holes 211 and 13 to pivotally secure the U-lugs 21 to the bands 11 of the headpiece 1 (see FIG. 4). Thereafter, the connection endpieces 222 of the connecting strips 22 of the connecting devices 2 are respectively inserted into the insertion holes 311 of the earmuff cups 3 to force the retaining ribs 223 of the respective connecting strips 22 into engagement with the respective engagement holes 312 of the earmuff cups 3 (see FIG. 5).

Further, the cross section of each retaining rib 223 is a regular triangle, having a front guide slope 2231 and a rear upright stop edge 2232. By means of the front guide slopes 2231, the retaining ribs 223 can conveniently be forced into the engagement holes 312. After insertion of the retaining ribs 223 into the associating engagement holes 312, the rear upright stop edge 2232 of each retaining rib 223 is stopped against one side of the associating engagement hole 312 to prohibit backward displacement of the respective retaining rib 223 relative to the associating engagement hole 312, and therefore the earmuff cups 3 are respectively and positively secured to the connecting strips 22 of the connecting devices 2.

Referring to FIGS. 1˜3 again, when the two bands 11 are coupled together, the user can move the two bands 11 relative to each other to adjust the length of the headpiece 1. When adjusting the length of the headpiece 1, the sliding grooves 14 of one band 11 guide sliding movement of the coupling block 12 of the other band 11. Further, each band 11 has a thickness gradually reducing in direction from the coupling block 12 toward the through hole 13, i.e., from the second end toward the first end. This band thickness design enables the headpiece 1 to be comfortably attached to the wearer's head. Further, by means of matching the recessed sliding grooves 14 of one band 11 with the protruding coupling block 12 of the other band 11, the two bands 11 are matched smoothly in a flat manner.

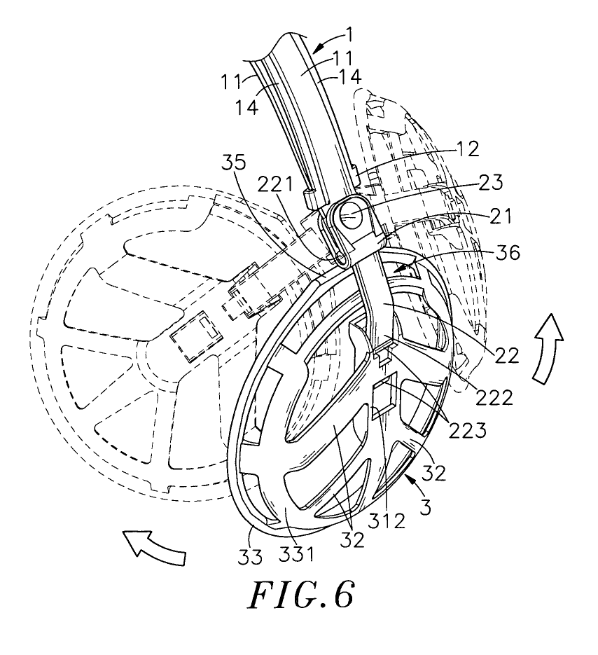

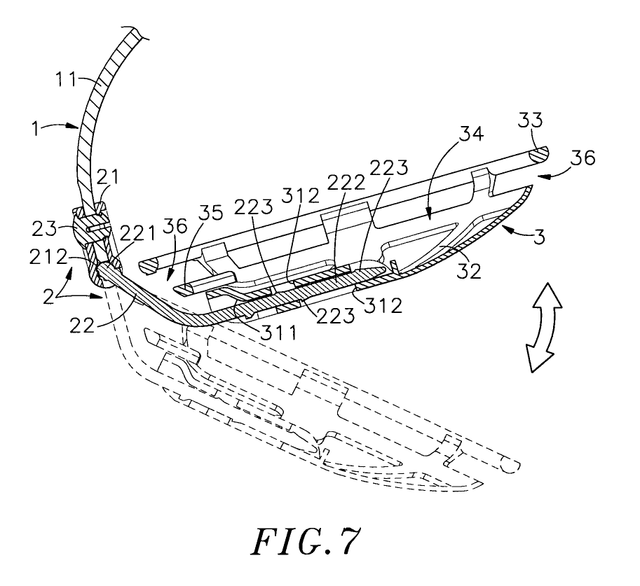

Referring to FIGS. 6 and 7, the U-lugs 21 of the connecting devices 2 are respectively pivotally connected to the through holes 13 of the bands 11 by the respective pivot bolts 23, and therefore the earmuff cups 3 can be biased with the connecting devices 2 forwards and backwards relative to the headpiece 1. When biasing the earmuff cups 3 with the connecting devices 2 forwards and backwards relative to the headpiece 1, the chamfered end edge 15 of the first end of each band 11 is rubbed against the associating U-lug 21. After adjustment, the chamfered end edge 15 of the first end of each band 11 is stopped against the inside wall of the associating U-lug 21, stopping the earmuff cups 3 in one of three positions, the middle position in line with the headpiece 1, namely, the front position perpendicular to the headpiece 1, and the rear position perpendicular to the headpiece 1 (see FIG. 6). Further, the coupling between the pivot axle 221 of the connecting strip 22 of each connecting device 2 and the bottom pivot housing 212 of the respective U-lug 21 allows the earmuff cups 3 to be biased with the associating connecting strips 22 relative to the associating U-lugs 21 and the headpiece 1 in direction away from or toward the wearer's head (see FIG. 7). Therefore, the wearer can adjust the earmuff device to fit the head comfortably.

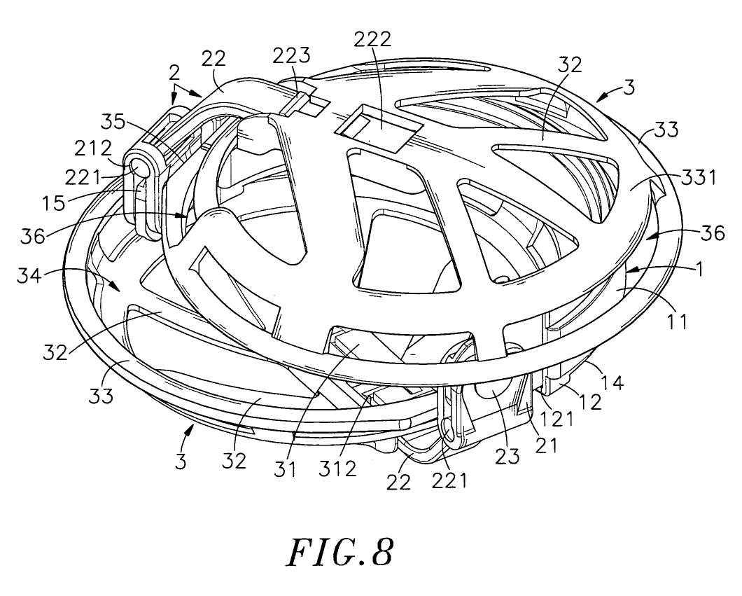

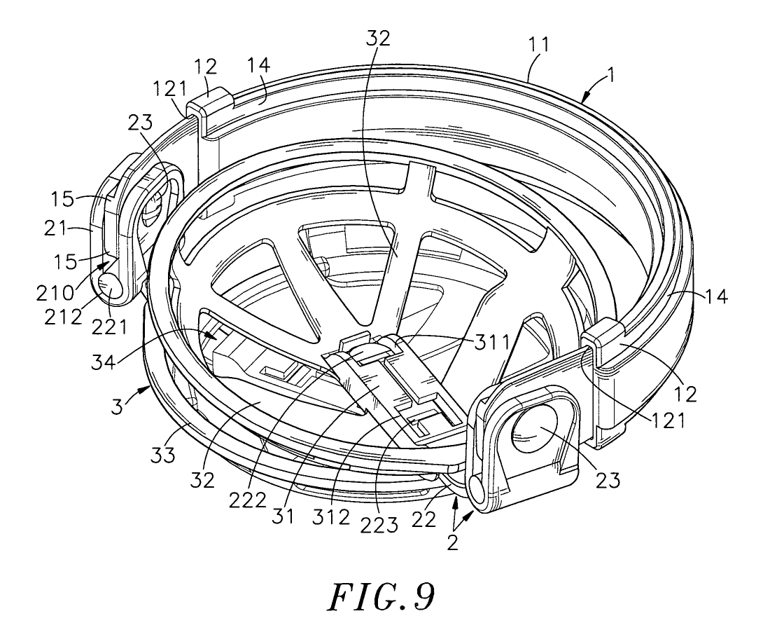

Referring to FIGS. 8 and 9, when not in use, the two bands 11 are moved toward each other to shorten the length of the headpiece 1, and then the connecting device 2 are turned to adjust the earmuff cups 3 relative to the headpiece 1 and closely attached to each other in reversed directions (see FIG. 8). Alternatively, the connecting device 2 are turned to adjust the earmuff cups 3 relative to the headpiece 1 and closely attached to each other in one same direction (see FIG. 9). Therefore, the earmuff device can be adjusted to fit the wearer's head comfortably. When not in use, the earmuff device can be collapsed and received in a compact condition, saving much the storage space.

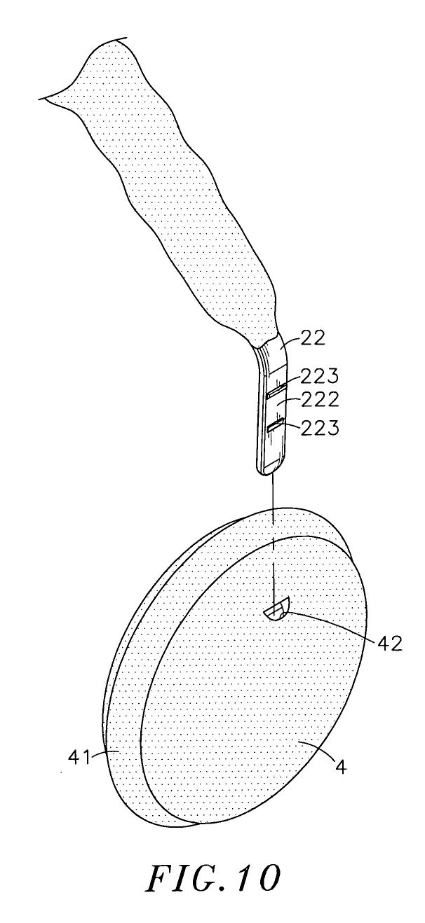

Referring to FIG. 10 and FIG. 1 again, each earmuff cushion 4 covers one earmuff cup 3 and a part of the connecting strip 22 of the associating connecting device 2 for contact with the side of the wearer's bead. Further, the bands 11 of the headpiece 1 and the connecting devices 2 may be respectively covered with soft covering means. According to the embodiment shown in FIG. 10, the earmuff cushion 4 is comprised of two fabric panels respectively attached to the front and back sides of the earmuff cup 3 and a binding strip 41 stitched to the two fabric panels. Further, the earmuff cushion 4 has an opening 42 corresponding to the insertion hole 311 of the associating earmuff cup 3 for the insertion of the associating connecting strip 22.

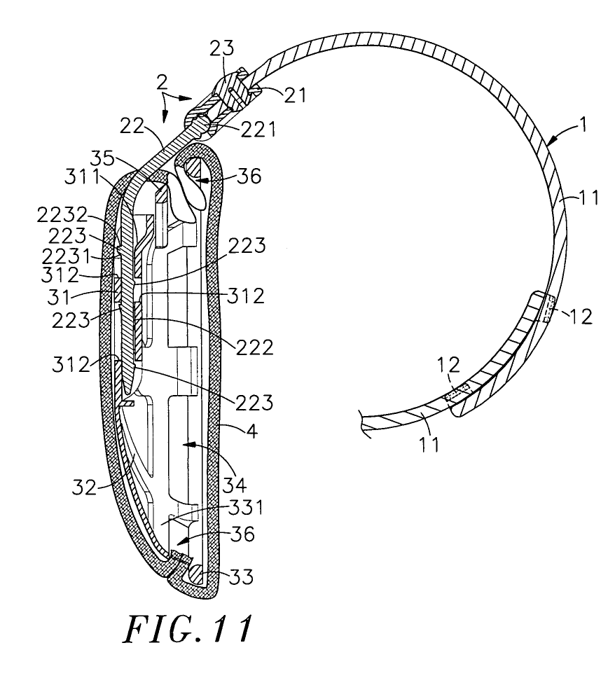

FIG. 11 shows an alternate form of the earmuff cushion 4. According to this embodiment, the earmuff cushion 4 is made in the form of a fabric pocket for receiving one earmuff cup 3. The fabric pocket can be formed by means of stitching two fabric panels together, and then turning the fabric pocket inside out to have the earmuff cup 3 be received inside the fabric pocket and also to have the stitches be kept from sight. After installation, the cuff around the opening of the fabric pocket is turned inwards toward the inside of the fabric pocket and inserted into one locating hole 36 of the earmuff cup 3.

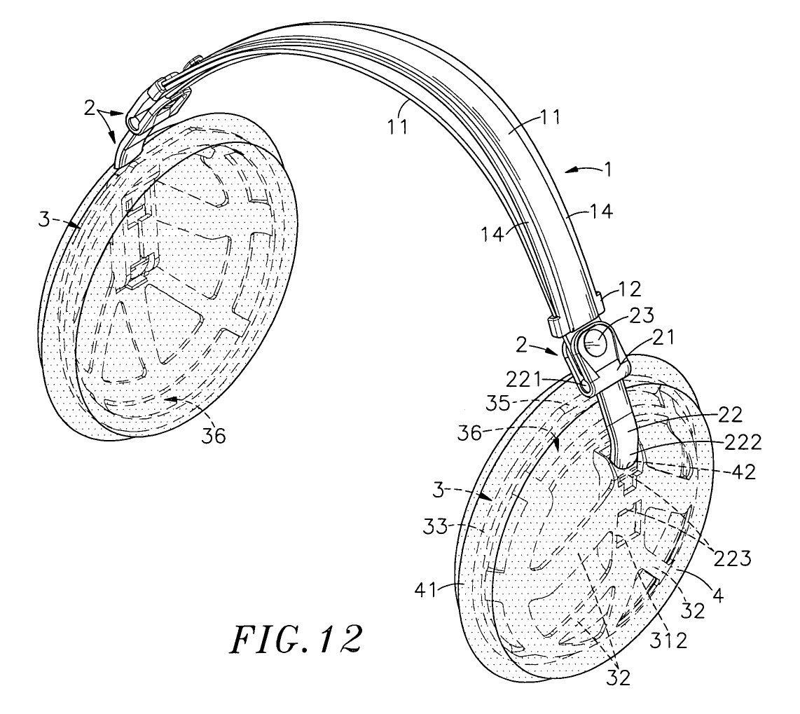

FIG. 12 is a perspective view of the present invention, showing the earmuff cushions 4 respectively covered on the earmuff cups 3, and the connecting strips 22 of the connecting devices 2 are respectively inserted into the openings 42 of the earmuff cushions 4 and connected to the associating earmuff cups 3.

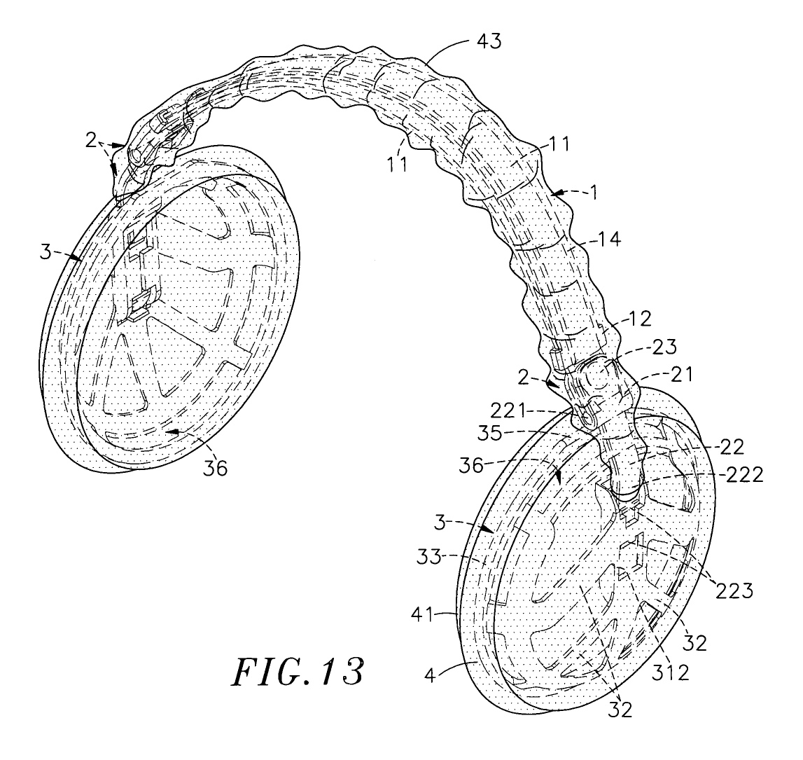

Referring to FIG. 13, a fabric sleeve 43 may be sleeved onto the headpiece 1 and connected between the two earmuff cushions 4. The fabric sleeve 43 has a certain length that allows adjustment of the length of the headpiece 1 to a certain extent.

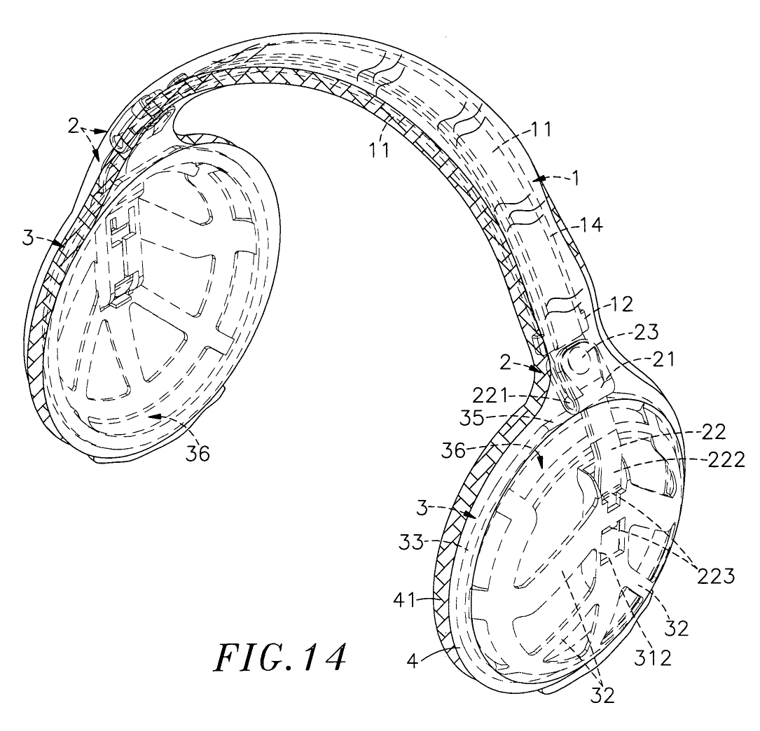

FIG. 14 shows still another alternate form of the present invention. This embodiment is substantially similar to the embodiment shown in FIG. 13 with the exception that the fabric sleeve 43 and the earmuff cushions 4 are made in integrity, i.e., two fabric panels are attached to the headpiece 1, the connecting devices 2 and the earmuff cups 3 from two opposite sides and then peripherally stitched together. Alternatively, a fabric material can be used to make a pocket subject to the configuration of the earmuff device, and then the earmuff device is inserted into the pocket, and then the cuff around the opening of the pocket is received together and inserted into one locating hole 36 of one earmuff cup 3.

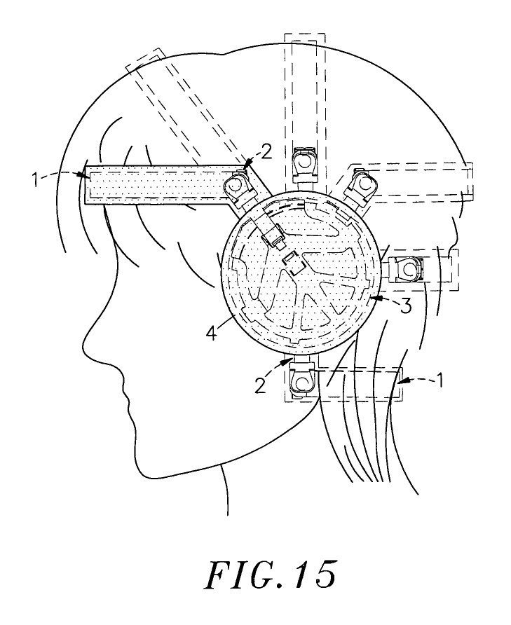

Referring to FIG. 15 and FIG. 1, when a person is wearing the earmuff device of the present invention, the wearer can adjust angular position of the headpiece 1 relative to the earmuff cups 3. For example, the headpiece 1 can be attached to the wearer's forehead, the top or back of the wearer's head or the back of the wearer's neck, while the earmuff cups 3 are attached to the wearer's ears, i.e., the user can freely adjust the length of the headpiece 1 and the angle of the earmuff cups 3 relative to the headpiece 1 as desired when wearing the earmuff device.

Although particular embodiments of the invention have been described in detail for purposes of illustration, various modifications and enhancements may be made without departing from the spirit and scope of the invention.

What the invention claimed is:

1. An earmuff device, comprising a headpiece, two earmuff cups, two connecting devices respectively connecting said earmuff cups to said headpiece, and two earmuff cushions respectively covered on said earmuff cups, wherein:
said headpiece is comprised of two smoothly arched bands that are overlapped on each other and axially slidable relative to each other, each said band having a first end, a second end opposing said first end, a through hole at said first end, and a coupling block disposed at said second end, an insertion slot defined in said coupling block, and two sliding grooves bilaterally extending along the length between said first end and said second end, said first end of one said band being inserted through said insertion slot of the other said band to have said coupling block of one said band be slidably coupled to said sliding grooves of the other said band;
each said connecting device comprises a U-lug attached to said first end of one said band of said headpiece, said U-lug having two through holes disposed at two sides and aligned in line, a pivot member fastened to said through holes of said U-lug and said through hole of one said band of said headpiece to pivotally secure said U-lug to said associating band and a pivot housing at a bottom side thereof, and a connecting strip connecting said U-lug to one said earmuff cup, said connecting strip having a pivot axle transversely disposed at one end thereof and pivotally coupled to said pivot housing of said U-lug and a connecting endpiece disposed at an opposite end thereof, said connecting endpiece having at least one retaining rib for engagement with one said earmuff cup;
each said earmuff cup comprises a cup base, an insertion hole defined in said cup base for the insertion of said connecting strip of one said connecting device, at least one engagement hole cut through said cup base and extending across said insertion hole of said respective earmuff cup for engagement with said at least one retaining rib of said connecting endpiece that is inserted into said insertion hole of said respective earmuff cup, a gradual enlargement cup rim, a plurality of radial ribs radially connected between said cup base and said gradual enlargement cup rim, a plurality of smoothly arched reinforcing ribs connected with said radial ribs, an accommodation space surrounded by said radial ribs, said cup base, said cup rim and said smoothly arched reinforcing ribs for receiving one ear of a person wearing the earmuff device, a plurality of locating holes defined between said cup rim and said smoothly arched reinforcing ribs and separated by said radial ribs.

2. The earmuff device as claimed in claim 1, wherein said first end of each said band of said headpiece has a chamfered end edge kept in friction engagement with said U-lug of one said connecting device.

3. The earmuff device as claimed in claim 1, wherein each retaining rib of said connecting endpiece of each said connecting device has a cross section shaped like a regular triangle and defining a front guide slop and a rear upright stop edge.

4. The earmuff device as claimed in claim 1, wherein each said earmuff cushion is comprised of two fabric panels respectively attached to front and back sides of one said earmuff cup and a binding strip stitched to said two fabric panels.

5. The earmuff device as claimed in claim 1, wherein each said earmuff cushion is made in the shape of a pocket that receives one said earmuff cup, said pocket being formed of two fabric panels stitched together and having an opening for the insertion of said associating earmuff cup and a cuff around the opening, said cuff being turned inwards toward the inside of said pocket and inserted into one locating hole of said received earmuff cup.

6. The earmuff device as claimed in claim 1, further comprising a soft sleeve sleeved onto said headpiece and connected between said earmuff cushions.
